(19) United States
(12) Patent Application Publication
(10) Pub. No.: US 2013/0202479 A1
(43) Pub. Date: Aug. 8, 2013

(54) PLASMA STERILIZER, PLASMA STERILIZATION SYSTEM, AND PLASMA STERILIZATION METHOD

(71) Applicants: Takumi Tandou, Hachioji (JP); Nobuyuki Negishi, Tokyo (JP); Naoshi Itabashi, Hachioji (JP)

(72) Inventors: Takumi Tandou, Hachioji (JP); Nobuyuki Negishi, Tokyo (JP); Naoshi Itabashi, Hachioji (JP)

(21) Appl. No.: 13/823,122

(22) PCT Filed: Oct. 21, 2010
(86) PCT No.: PCT/JP2010/068550

§ 371 Date: Apr. 8, 2013

(51) Int. Cl. A61L 2/20

(52) U.S. Cl. CPC A61L 2/20
USPC 422/3; 422/111

(57) ABSTRACT

An apparatus which determines activeness/inactiveness of bacteria in real time by measuring a specific light emission spectrum upon performing sterilization using plasma to highly efficiently sterilize is provided. As solving means, plasma is irradiated on a processing target from a plasma source connected to an alternate-current power supply and light emission of the processing target caused by the irradiation of plasma is detected by a light emission intensity detector unit. Particularly, by detecting wavelength intensity of hydrogen or hydroxyl group, activeness/inactiveness of bacteria can be determined at an early stage. Thus, an appropriate output of a power supply for sterilization can be controlled.

### TECHNICAL FIELD

The present invention relates to a plasma sterilization apparatus which inactivates adhesive bacteria and floating (airborne) bacteria in facilities and space such as bioclean rooms (herein after, referred to as BCR) requiring removal of microorganisms; more particularly, the present invention relates to monitoring technology capable of detecting activeness or inactiveness of bacteria in real time.

### BACKGROUND ART

Expectations have been raised for achieving regenerative medicine using artificially cultured cells and tissues to regenerate damaged skin, cornea, internal organs, etc. for functional recovery of patients. The number of patients having target diseases is expected to be 20,000 per year even when only those having cornea regeneration are considered and thus practical application of technology has been longed for. It is expected that participation of pharmaceutical companies will also become obvious in the future and regenerative medicine will grow into a new medical industry.

BCR, in which aseptic manipulation can be carried out, is essential in clinical studies and thus establishment of sterilization techniques for surface adhesive bacteria has been an important problem to maintain the indoor environment in the BCR. Conventional sterilization has been carried out by formalin fumigation inside a room but that usage has become prohibited because it is harmful to human body since its carcinogenicity is pointed out. Therefore, other adhesive bacteria sterilization techniques substituting formalin are desired.

To study on a novel sterilization method of surface adhesive bacteria inside a BCR, sterilization methods of surface adhesive bacteria which have been generally used in medical practice or medical-related manufacturers have been researched and roughly classified as follows.

i) Sterilization methods by heating such as dry-heat sterilization, high-pressure steam sterilization, and boiling water sterilization;

ii) Radiation sterilization methods by radiation (γbeam etc.), ultraviolet rays (near 254 nm wavelength), electron beam, etc.; and

iii) Gas sterilization methods by ethylene oxide gas, hydrogen peroxide gas, etc.

Although there are various sterilization methods depending on material, shape, etc. of the sterilized subject as exemplified above, application of the sterilization methods mentioned above in a BCR is considered to be difficult. For example, since the floor in a BCR is a resin-based material, the heat sterilization methods which raise temperature to about 120° C. cannot be used. Also, the process time is a problem in the radiation sterilization methods because its sterilization ability is low and thus radiation for several tens of minutes to several hours is required.

With regard to the gas sterilization methods, since they are harmful to human body same as formalin and require several hours to one day for degassing, using the gas sterilization methods has become avoided. Against such a background, sterilization methods using plasma as a novel sterilization method capable of low-temperature and high-speed processing and not using harmful substances have been getting attentions.

### PRIOR ART DOCUMENTS

### Patent Documents

Patent Document 1: Japanese Patent Application Laid-Open Publication (Translation of an International Application) No. 2009-545673
Patent Document 2: Japanese Patent Application Laid-Open Publication (Translation of an International Application) No. 2008-525750
Patent Document 3: Japanese Patent Application Laid-Open Publication No. 2007-117254

### DISCLOSURE OF THE INVENTION

### Problems to be Solved by the Invention

When performing the sterilization treatment in a BCR, a cultivation test using culture media is generally used as a method for determining presence and activeness/inactiveness of bacteria; however, the determination requires time for about several days. Therefore, it is impossible to know when and where contamination due to bacteria occurs in the BCR in real time and thus, currently, the sterilization process of the whole room by formalin has been empirically carried out in a cycle of once every few weeks.

As described above, there is a tendency that fumigation inside a room by formalin is prohibited because formalin is harmful to human body and substitute means such as plasma have been studied. However, it is impossible to irradiate plasma on the whole BCR room at one time, and thus it will be possible to sterilize the whole BCR room effectively if existing position of bacteria is detected and irradiated and the irradiation time is decided when inactiveness is determined.

If such sterilization is achieved, only specific portions where bacteria are increased in a BCR need the sterilization work and it will become unnecessary to make a complete stop (all the workers are evacuated) of the BCR for a few days for sterilization. In addition, it is possible to increase power for plasma generation only at a portion where bacteria exist and thus it will be possible to carry out sterilization on the whole BCR room with low power.

As a method of determining inactivation of bacteria in real time, Patent Document 1 discloses a method of measuring oxygen radicals in the plasma treatment using an optical detector. Changes of oxygen radicals desorbed from bacteria are observed by plasma and determination of extinction of bacteria is made when the changes of oxygen radicals become constant as bacteria are completely disappeared (bacteria are decomposed by various radicals generated by the plasma. The smaller the radius of bacteria by the decomposition, the smaller the amount of oxygen radicals desorbed; thus, timing at which the amount of generated oxygen radicals becomes constant when bacteria are completely disappeared).

However, although it is possible to determine disappearance of bacteria in the method described above, inactivation of bacteria which have been already generated before disappearance cannot be determined. Therefore, irradiation of plasma until bacteria are completely disappeared poses an increase in the process time. In addition, considering that the usage of the above-described method for the sterilization in a BCR, since a floor and walls inside the BCR are organic substance (main components: C, O, N), oxygen radicals may be desorbed from the floor and walls by irradiation of plasma; thus, it is expected to be difficult to determine the disappearing time of bacteria.

Patent Document 2 discloses a system, as an apparatus for monitoring dehydration operation during a freeze-drying process, of determining whether water (moisture) inside a chamber is completely dehydrated or not by generating plasma inside the chamber and paying attention to hydrogen radicals in the emission spectrum of the plasma. It is also disclosed that there is a sterilization effect as OH radicals are generated by generating plasma in a state that water exists inside the chamber.

However, the above-described way is originally a system for measuring the amount of water (=humidity) presenting inside the chamber for monitoring the dehydration state inside the chamber; thus, it is not a system of determining inactiveness by measuring reactive products generated from bacteria. In addition, although OH radicals are generated by generating plasma and thus an effect of sterilizing bacteria can be expected, water is detected when a large amount of water is contained in a gas for plasma generation when and thus it is difficult to measure spectrum of hydrogen desorbed from bacteria.

Patent Document 3 discloses, focusing on a light emission phenomenon correlated to plasma discharge, an air-cleaner apparatus capable of effectively controlling the generated amount of ions by estimating the generated amount of positive and negative ions based on the intensity of emission of light generated by the plasma discharge phenomenon. The emission intensity in a surface of ion-generating electrodes (plasma generating portions) is monitored and an output (generated amount of ions) of the ion-generating electrodes can be controlled based on detected emission information.

However, the above-described methods correspond to temporal changes of the electrodes and humidity changes in the discharge space by detecting light emission amount from the plasma and thus they cannot determine inactiveness of bacteria by taking notice of a specific emission spectrum.

FIG. 9 is a diagram studied by the inventors of the present invention in advance for an early determination of inactiveness of a subject organism to be processed. While there has been an apparatus of determining light emission intensity of carbon (C₂) in this art, there have been strong demands of carrying out the sterilization process in a short time with suppressing the irradiation time of plasma.

As one example of the experiments made by the inventors, a case of yeast will be explained. Yeast forms tissues in a shell-like shape outside the cell cytoplasm and exhibits high resistant characteristics against sterilization by heat and ultraviolet rays. When Bacillus subtilis is irradiated with plasma, its outer shell is first altered and then its internal cell is altered. The inventors have taken attention to a phenomenon of increasing the light emission intensity of C₂ around timing at which light emission of H attenuates like that in FIG. 9. This indicates that hydrogen is withdrawn in advance at an initial stage and carbon is withdrawn at the next stage. Then, the sterilization process is ended at timing at which the light emission intensity of hydrogen is increased; then, inactiveness of bacteria was confirmed when a method of cultivating the subject by a culture sheet was used. From this result, a conclusion was made that performing monitoring of a specific emission spectrum (=light emission intensity) capable of detecting inactiveness earlier than carbon is favorable to determine inactiveness of bacteria early.

Therefore, a preferred aim of the present invention is to provide a plasma sterilizer capable of highly efficient sterilization by determining presence and activeness/inactiveness of bacteria in real time by measuring a specific light emission spectrum of a component derived from an organism when performing sterilization using plasma.

### Means for Solving the Problems

To solve the above-mentioned problems, a plasma sterilizer of the present invention includes: a power supply outputting alternating-current voltage; a plasma source driven by the power supply; a light emission intensity detector detecting light emission intensity of hydrogen or hydroxyl group from a region in which a gas that is radicalized by the plasma source is present; and a controller controlling an output of the power supply based on the light emission intensity.

In addition, to solve the above-mentioned problems, a plasma sterilizer system of the present invention includes: a power supply outputting alternating-current voltage; a plasma source driven by the power supply; a light emission intensity detector detecting light emission intensity of hydrogen or hydroxyl group from a region in which a gas that is radicalized by the plasma source is present; a clock defining a detection time of the light emission intensity; and a controller controlling an output of the power supply based on the light emission intensity within a certain period measured by the clock.

Moreover, to solve the above-mentioned problems, a method of plasma sterilization of the present invention includes: a power supply outputting alternating-current voltage; a plasma source driven by the power supply; a light emission intensity detector detecting light emission intensity; and a controller performing control of changing an output of the power supply, the method including: a first step of applying the output of the power supply to the plasma source; a second step of generating a gas that is radicalized by the plasma source; a third step of detecting light emission intensity of hydrogen or hydroxyl group from a region in which the gas is present; and a fourth step of controlling the output of the power supply based on the light emission intensity.

### Effects of the Invention

According to the present invention, it is possible to highly efficiently sterilize upon a sterilization process using plasma.

### BRIEF DESCRIPTIONS OF THE DRAWINGS

FIG. 1 is a schematic diagram illustrating a configuration of a plasma sterilizer according to the present invention;

FIGS. 2A to 2C are explanatory diagrams of a detecting method of a target processing organism according to the present invention;

FIG. 3 is a schematic diagram illustrating another example of a configuration of the plasma sterilizer according to the present invention;

FIG. 4 is a schematic diagram illustrating a still another example of a configuration of the plasma sterilizer according to the present invention;

FIG. 5 is a schematic diagram illustrating a configuration of the plasma sterilizer and a target processing surface according to the present invention;

FIGS. 6A and 6B are schematic diagrams illustrating a self-moving plasma sterilizer according to the present invention;

FIG. 7 is a schematic diagram in which the plasma sterilizer according to the present invention is embedded in a whole BCR system;

FIG. 8 is a schematic diagram illustrating a plasma sterilizer for floating bacteria according to the present invention; and

FIG. 9 is a schematic diagram in which periodic transitions of light emission intensity of hydrogen and carbon are compared according to the present invention.

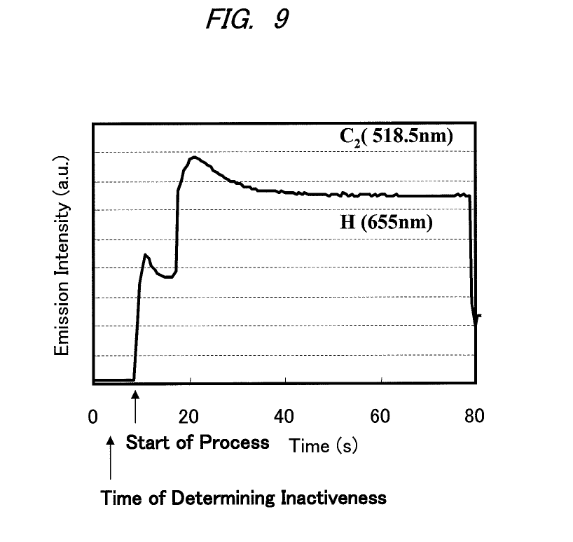

### BEST MODE FOR CARRYING OUT THE INVENTION

### First Embodiment

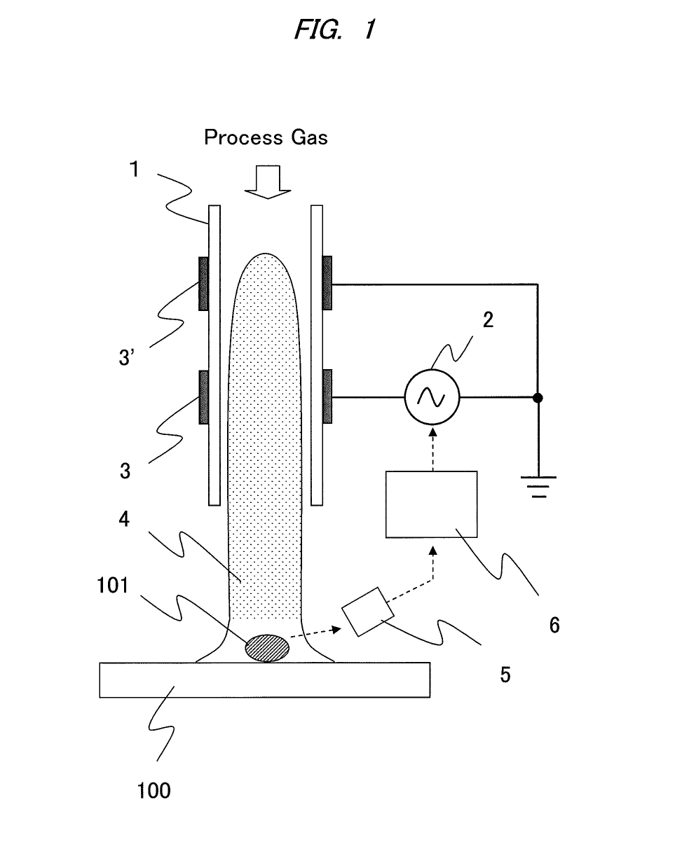

FIG. 1 is a schematic diagram illustrating a configuration of a plasma sterilizer according to the present invention. Under the atmospheric pressure, there is a plasma source to which a process gas is supplied. In the plasma source, a high-frequency electrode 3 to which power is applied from a high-frequency power supply 2 and a ground electrode 3′ are provided and the plasma source generates plasma 4 inside a tube of an insulator 1 to irradiate plasma to a processing target organism 101 that is on a processing target surface 100.

Incidentally, the irradiation of the plasma as referred to herein is gas generated upon discharge and it is directed to a state in which freely moving charged particles are present and electrically neutral. That is, phenomena not only the discharging portion directly works on bacteria but also radicals generated by the discharge gives a sterilization effect on bacteria are included. Thus, the sterilization process can be performed when a generation region of radicals, instead of a discharge region, is present in the processing target surface 100.

When performing sterilization inside a BCR, the target processing surface 100 is a floor and walls of the BCR and the target processing organism 101 is, for example, Bacillus subtilis.

By using or adding oxygen as a gas for generating the plasma 4, oxygen radicals are generated in the plasma 4. When the target processing organism 101 is irradiated with the plasma 4, desorption of hydrogen from the surface from cell walls of the target processing organism 101 by oxygen radicals is started. In this manner, the target processing organism 101 is inactivated as protein of the surface is altered.

Upon the start of the desorption, by measuring a light emission spectrum (e.g., 655 nm) of hydrogen in the plasma 4 by a spectrometer 5, a start time and an end time of the hydrogen desorption can be detected. When the hydrogen desorption ends, that is, when the light emission is hydrogen is attenuated and the light emission amount becomes constant, the target processing organism 101 is inactivated; if the hydrogen desorption can be detected, inactivation of the target processing organism 101 can be determined.

Detected information of the light emission intensity of hydrogen from the spectrometer 5 is transmitted to a control board 6 of the high-frequency power supply 2. When determining a presence of the target processing organism 101, the output power of the high-frequency power supply 2 may be set at a low level to reduce power consumption. Then, when a presence of the target processing organism 101 is recognized, the output power of the high-frequency power supply 2 is raised until inactivation of the target processing organism 101 is confirmed. In this manner, the generated amount of oxygen radicals in the plasma is increased to inactivate the target processing organism 101 at high speed.

Note that, when monitoring the target processing organism 101 by detecting a light emission spectrum of hydrogen, water may be detected if water etc. is attached to, for example, the target processing surface 100 in the BCR.

In this case, a light emission spectrum of a substance derived from an organism (e.g., phosphorus) may be detected together with the light emission spectrum of hydrogen. Phosphorus is a component contained in lipids of organisms and not contained in water or other organic substances. That is, presence of the target processing organism 101 may be determined by detecting the light emission spectrum of phosphorus to determine inactiveness of the target processing organism 101 from the light emission spectrum of hydrogen.

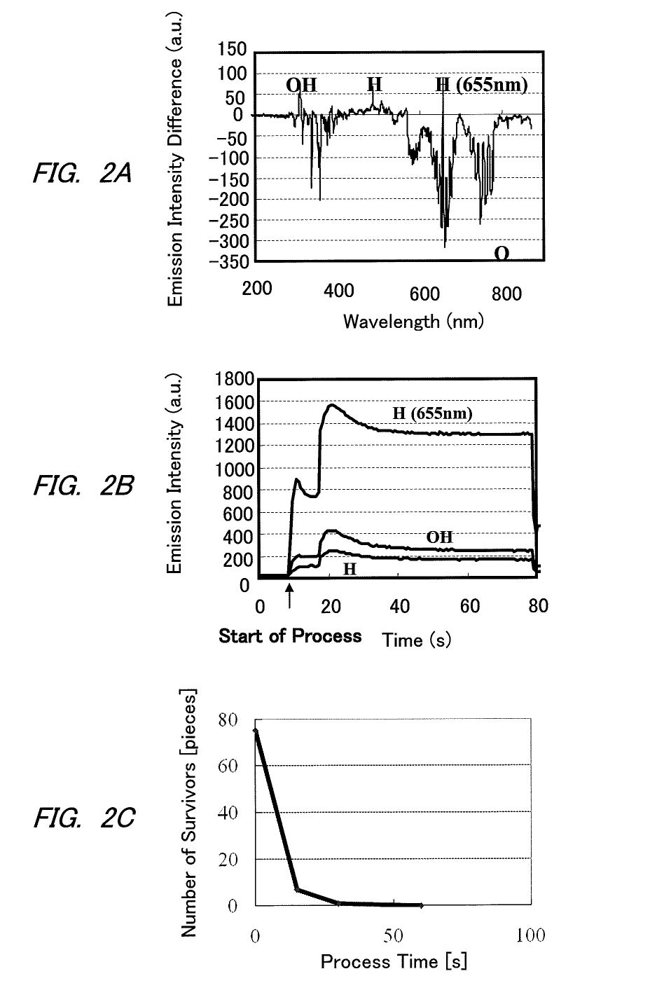

FIGS. 2A to 2C are explanatory diagrams of a method of detecting the target processing organism according to the present invention.

FIG. 2A is a measurement result of a light emission spectrum in the case of using the air as a processing gas and using yeast (Saccharomyces cerevisiae) as a target processing organism. The plot shows a wavelength on the horizontal axis and a difference in light emission intensity on the vertical axis. The difference in light emission intensity means a subtraction of a wavelength in the case without a presence of yeast from a wavelength in the case with a presence of yeast. Oxygen is contained by about 20% in the air that is a process gas and thus a light emission peak of hydrogen can be detected as hydrogen in the yeast surface is desorbed by oxygen radicals in the plasma. While the value of oxygen radicals exhibits a negative value on the other hand, this is because oxygen radicals are consumed upon desorption of hydrogen etc. Note that, while the providing area of yeast here is about 2% of the area where the plasma is irradiated, the light emission of hydrogen can be sufficiently detected.

FIG. 2B illustrates how the light emission intensity of hydrogen of which a light emission peak has been detected is changed with time. After the processing is started (start of plasma irradiation), it can be understood that desorption of hydrogen is started by oxygen plasma and the desorption of hydrogen is attenuated from about 30 seconds of processing time and then the intensity of the light emission spectrum of hydrogen (e.g., 655 nm) becomes constant. It means a state in which light emission from the desorption of hydrogen is weakened and only emission of plasma caused by the apparatus operation is detected. That is a state in which the light emission intensity is constant. Also, in a state at the timing of 80 (seconds) in FIG. 2B, the alternating current power supply is deactivated. As the light emission intensity changes over time in this manner, it is suitable to perform control with defining that a certain value at which the state is recognized to have been changed as a threshold value.

Further, although not illustrated here, when hydrogen of the outermost surface of yeast is desorbed, carbon etc. are desorbed subsequently. At this timing, yeast has been already inactivated and it is unnecessary to perform a sterilization process.

In the present invention, focusing on the fact that bacteria is inactivated at the timing at which hydrogen in a surface is desorbed, a light emission spectrum of hydrogen or hydroxyl (OH) is measured. Thus, inactivation of survivor bacteria can be determined earlier than monitoring desorption of carbon and thus a reduction of the process time and an improvement of process efficiency are achieved.

FIG. 2C illustrates a relationship of the irradiation time of plasma and the number of survivor bacteria. Yeast after respective process time periods were cultured on a culture medium and the number of survivor bacteria was measured. As a result, inactivation of yeast can be also confirmed at the same time of desorption of hydrogen by oxygen radicals in the plasma.

Note that, while FIGS. 2A and 2B are measurement results of light emission spectra in vacuum, the same spectrum measurement is also available in the atmosphere.

Note that, while a series of descriptions has been made in FIG. 1 and FIGS. 2A to 2C, each configuration will be further described.

The insulator 1 relates to characteristics of the generated plasma. When the plasma is generated by performing an atmospheric discharge from electrodes, an arc high-current discharge is made. However, a low-current glow discharge can be performed by using the insulator 1 and thus power can be reduced. Thus, the insulator 1 is provided to reduce the power of discharge and so the insulator 1 is not always necessary upon working of the present invention. Also, the volume of the discharge space can be reduced in glow discharge more than in other discharge systems and thus is suitable to small-sized apparatuses like the present invention.

The high-frequency power supply 2 controls the potential and frequency required in discharge. By a basic operation, the speed of inactivation of the target processing organism can be increased by increasing the magnitude of the potential and frequency. In addition, when a presence of the target processing organism 101 is not confirmed, the magnitude of the potential and frequency may be reduced. Further, the control may be performed with a potential and a frequency at which a low-current glow discharge can be generated even in the absence of the insulator 1. For example, a high-frequency voltage is controlled in discrete pulses to suppress the amount of current flowing in the plasma. In this manner, since there is also a case of reducing power by the high-frequency power supply 2, the insulator 1 for power reduction can be omitted.

Changing the shapes of the high-frequency electrode 3 and the ground electrode 3′ can change characteristics of the generated plasma. By changing electrode shapes, power of the apparatus can be reduced in the same manner as the insulator 1 and the high-frequency power supply 2 described above.

Note that the plasma is generated in accordance with an electric field formed by the high-frequency electrode 3 and the ground electrode 3′. Thus, when the high-frequency electrode 3 and the ground electrode 3′ are arranged to be close to the target processing surface 100, higher-density plasma can be irradiated on the target processing surface 100. However, the plasma may be at high temperature in some cases and thus a distance to some extent not posing a temperature degradation to the target processing surface 100 may be provided.

The example in which the target processing surface 100 is a floor of a BCR has been described. However, it is also compatible to sterilization of the walls by making a portion for performing sterilization crawl along wall surfaces. Other than making the apparatus itself moved along the sidewalls by magnetic force and adhesive force, the apparatus may be such that the portion on which the sterilized process is performed is moved along the wall surface.

The example that the target processing organism 101 is, for example, Bacillus subtilis has been described. The reason of exemplifying Bacillus subtilis is that Bacillus subtilis exhibits high resistance against sterilization by heat and ultraviolet rays and is thus used in biological indicators (BI) of this art. Thus, the present invention is also effective to bacteria having high resistance against sterilization by heat and ultraviolet rays and the range of bacteria which can be processed are wide in addition to Bacillus subtilis and yeast.

An example has been described that the gas for generating the plasma 4 is, for example, oxygen. However, it is not limited as long as the gas is for desorbing organic substances. The reason of selecting oxygen here is that oxygen exists in the atmosphere and also is highly effective in desorbing organic substances. By supplying oxygen from the atmosphere, it is not necessary for the plasma sterilization apparatus to take along a cylinder (tank) in which a desorbing gas is sealed and thus downsizing can be achieved. In addition, it is also unnecessary to replace the gas cylinder (tank) and thus there is an effect of reducing running costs.

Further, for forcible convection of the gas for generating the plasma 4 to the processing surface 100, a fan or the like for ventilation may be mounted. Depending on a presence or absence of a device for forcible convection and the strength of convention, more radicalized gas is present on the processing surface and thus the processing efficiency can be improved. In addition, when natural convection is utilized, while the processing efficiency is lowered than the case of having a device for forcible convention, the device for forcible convection can be omitted.

Inactivation of a target for detecting an intensity change in wavelength can be also determined even when a light emission spectrum of hydroxyl group (OH) is measured except for that of hydrogen. As illustrated in the detection result in FIG. 2B of hydroxyl group (OH), while the light emission spectrum of hydroxyl group (OH) has lower intensity than that of hydrogen, there is detection sensitivity sufficient to determine a presence of active or inactive bacteria.

A light emission spectrum of each substance means a range of wavelength in which a satisfactory light emission intensity of each substance such as hydrogen, hydroxyl group or phosphorus can be obtained. Generally known regions of wavelengths of respective substances include near 410 nm to 490 nm or near 650 nm to 660 nm having a peak at 656 nm for hydrogen. In addition, near 302.1 nm to 308.9 nm for hydroxyl group, and near 215.4 nm to 255.5 nm or 919.4 nm to 1058.2 nm for phosphorus are typical. In this manner, the detection may be performed with selecting from a wavelength range in which satisfactory light emission intensity of each substance can be obtained.

The spectrometer 5 performs detection of light emission intensity of a waveform at which light emission intensity of each substance can be well obtained, using a color filter and a light-receiving element, and thus it is not always necessary to detect the entire visible light region. Since it is only necessary to be able to detect intensity of a specific wavelength, downsizing and cost reduction of the apparatus can be achieved. Also, in addition to directing the light-receiving unit of the spectrometer 5 directly to the target processing surface 100, further better detection sensitivity can be obtained by using a condenser lens and/or optical fiber. Particularly, when an optical fiber is interposed between the target processing surface 100 and a light-receiving unit of the spectrometer 5, it is not necessary to install the spectrometer 5 in a vicinity of the target processing surface 100 and thus the degree of freedom can be increased.

Although not illustrated in FIG. 1, a suction unit for sucking inactivated bacteria and dust may be further attached. As well as inactivating bacteria, inactivated bacteria and dust being present around the inactivated bacteria are sucked and thus the cleanness inside the room can be improved and there is a synergetic effect with prevention of bacterial growth.

### Second Embodiment

A second embodiment of the present invention will be described hereinafter. Even when other plasma generating methods than that of the first embodiment is used, determination of presence and inactiveness of a target processing organism 101 by light emission spectrum of the present invention is possible.

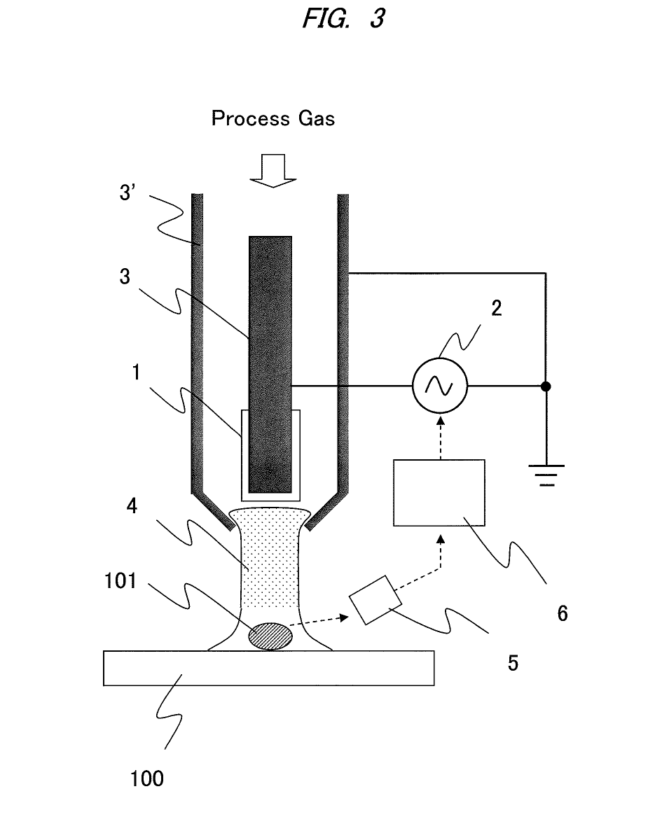

For example, FIG. 3 is a schematic diagram illustrating another example of the configuration of the plasma sterilization apparatus of the present invention. In the structure, a high-frequency electrode 3 and a ground electrode 3′ are facing each other and at least one of the electrodes is protected by an insulator 1. Plasma 4 is generated at a portion where a space between the facing electrodes is the narrowest and irradiated onto the target processing organic 101 that is on a target processing surface 100 along a flow of a process gas.

A spectrometer 5 and a control board 6 are installed in the same manner as the first embodiment and an output of a high-frequency power supply 2 is controlled based on intensity information of a light emission spectrum of hydrogen. Note that, when it is possible to suppress the amount of current flowing in the plasma by controlling high-frequency voltage supplied from the high-frequency power supply 2 in a discontinuous pulse form, the temperature of the plasma will not be high to some extent to degrade the target processing surface 100 inside the BCR even without the insulator for protecting the electrode.

By using the configuration of FIG. 3, the plasma discharge portion and the target processing organic 101 can be closer to each other than they are in FIG. 1. Thus, inactivation of the radicals in the plasma is further reduced and a low-power sterilization processing is available.

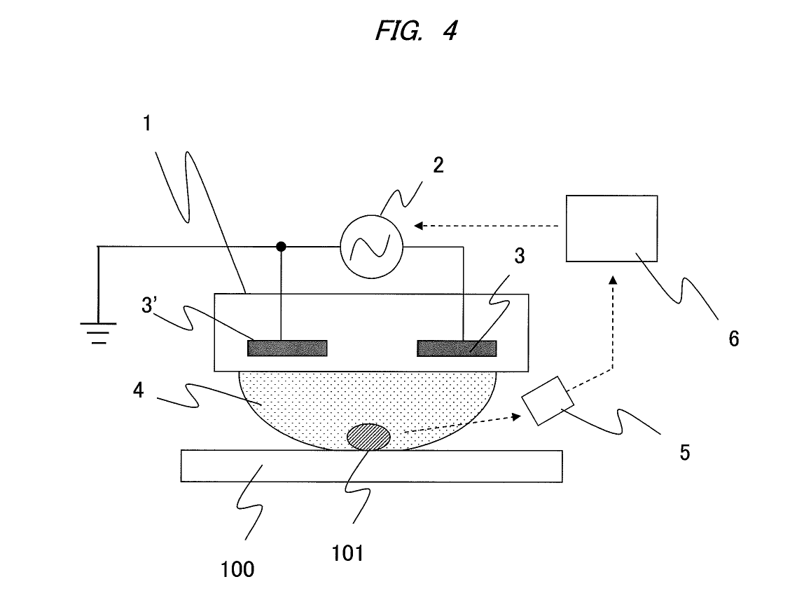

In addition, a schematic diagram to be still another example of the configuration of the plasma sterilization apparatus of the present invention is illustrated in FIG. 4. By providing a high-frequency electrode 3 and a ground electrode 3′ inside an insulator 1, plasma is generated in a vicinity of a surface of the insulator 1.

In the present structure, plasma is generated being stuck to the surface of the insulator 1 and thus the insulator 1 is put directly close to the target processing surface 100. In this manner, plasma can be generated in a large area and thus a wide area of the target processing surface 100 can be processed in a lump. A measurement method of light emission spectrum here is the same as that of FIG. 1.

### Third Embodiment

A third embodiment of the present invention will be described hereinafter.

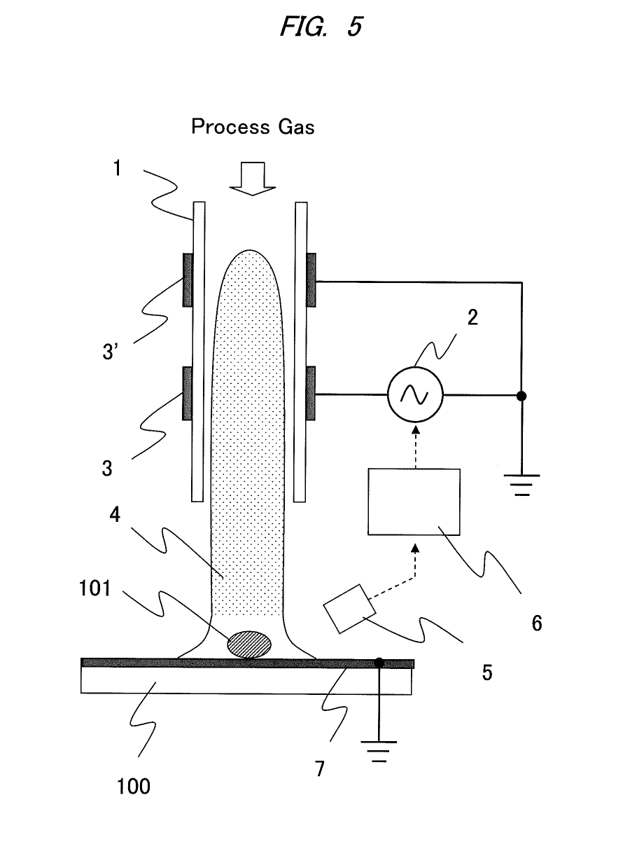

FIG. 5 is a schematic diagram illustrating a plasma sterilization apparatus and a configuration of a target processing surface of the present invention. By providing a ground electrode for target processing surface 7 to a topmost surface of a target processing surface 100, an electric field is formed between a high-frequency electrode 4 and the ground electrode for target processing surface 7. In this manner, plasma is accelerated in the electric field and collide with the target processing organism 101 and thus hydrogen desorption of the target processing organism by oxygen radicals is accelerated.

As a result, hydrogen radicals generated per a unit time are increased and light emission detection of hydrogen is made easier. Also, required time of inactivation can be also shortened.

### Fourth Embodiment

A fourth embodiment of the present invention will be described hereinafter.

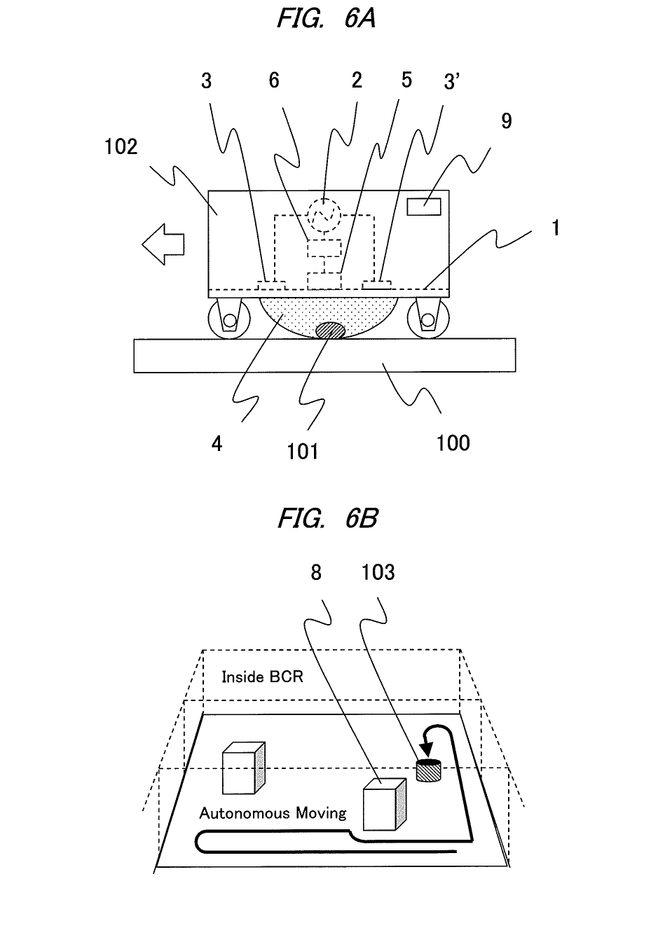

A self-moving plasma sterilization apparatus is illustrated in FIGS. 6A and 6B. As a typical system for attached bacteria sterilization, the plasma sterilization apparatus of either of FIG. 1 or FIGS. 3 to 5 of the present invention is mounted on a robot having a moving portion and the robot moves in a BCR as illustrated in FIG. 6A.

In FIG. 6A, an example of mounting the plasma sterilization apparatus of FIG. 4 on a self-moving robot is illustrated. Autonomous moving means moving indoors through a target avoiding obstacles 8 as illustrated in FIG. 6B. Here, an operation may be programmed such that, when the target processing organism 101 is searched for with generating plasma at a low power and the target processing organism 101 is found, the moving is stopped and an output of plasma generation is increased.

Further, during irradiation of plasma 4 to the target processing organism 101, the status in a sterilization processing may be displayed to the outside by display means 9 like LEDs etc. provided to the above-described apparatus 102. Further, the apparatus 102 may be back to a charging space installed in the BCR by autonomous moving after moving around in the whole BCR. Timing of operating the apparatus 102 is once in a few hours or once in a day depending on a required cleanness of the BCR.

Although it is possible to operate the apparatus 102 even while a worker(s) is at work, the apparatus 102 may be operated in night time when a worker(s) leaves from the BCR. In this manner, it is not necessary to completely stop the operation of the BCR for a few days for the sterilization processing and thus it is possible to keep the inside always clean.

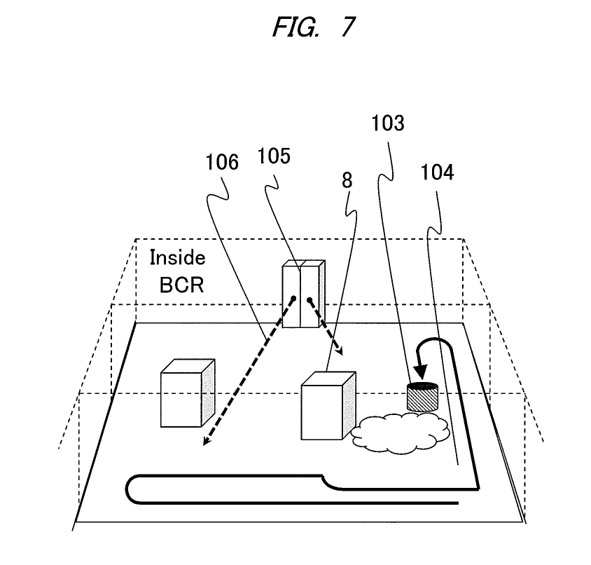

FIG. 7 is a schematic diagram in which a plasma sterilization apparatus is embedded in a whole BCR system with providing a logger function for storing detection information of light intensity inside the apparatus or outside the apparatus, i.e., not the apparatus body. By combining detection information of light intensity and time information separately prepared by a clock circuit etc., it is possible to store how much detection information of light intensity has been obtained in a certain time period. Thus, the level of contamination after operating for a certain time period on the field can be determined and an output power of a high-frequency power supply 2 and an operation cycle of the apparatus can be appropriately set.

In addition, by combining rotation information of a motor of a moving portion with the logger function, it is possible to map where contaminated parts are present on the field. For the mapping, a sensor for obtaining position information may be suitably provided. By comparing the mapping information thus obtained and a result of an arrangement plan of work tables and staff in the BCR, an easily contaminated location 104 or easily contaminated time can be specified depending on the arrangement. By performing the sterilization work with weighting assigned to the specified space and time, the BCR can be operated at a further lower contamination level.

Moreover, by performing control for suppressing factors of letting bacteria being present on the floor surface soar with respect to the specified space and time, possibility of attachment of bacteria onto samples in the BCR can be lowered. More specifically, control is performed so as not to cause convection of air in the specified location and time with respect to an air convection apparatus (e.g., air conditioning apparatus) 105. Alternatively, a display monitor or the like may be embedded in a system to display the specified location and time and an alarm function may be provided so that moving of measurement devices and a person(s) inside the BCR are deterred.

### Fifth Embodiment

A fifth embodiment of the present invention will be described hereinafter.

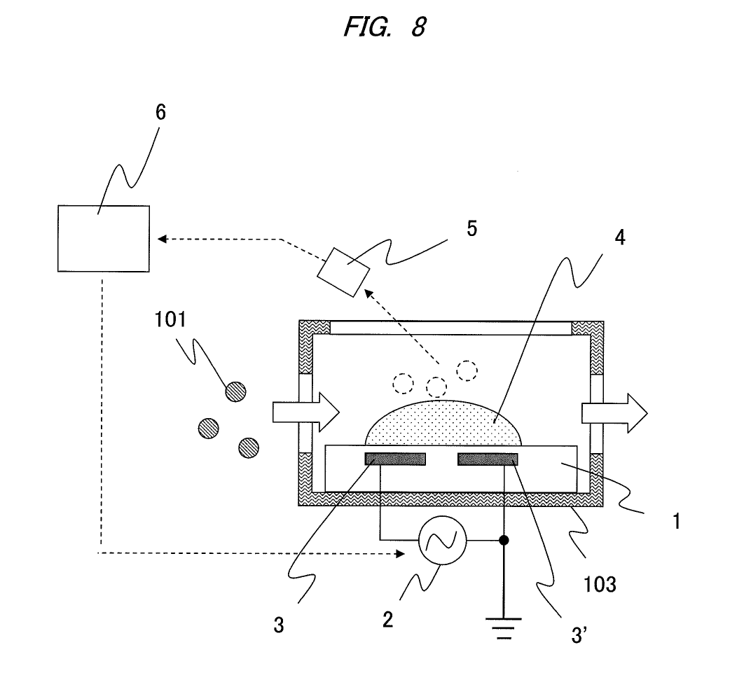

FIG. 8 is a schematic diagram illustrating a plasma sterilization apparatus for floating bacteria according to the present invention. In the cleaning of a BCR, in addition to a target processing organism to be attached on a floor and walls, inactivation of a target processing organism 101 floating in the air is also important.

For example, a plasma sterilization apparatus same as that in FIG. 4 is installed in a processing box 103 and a light emission spectrum of plasma inside the processing box 103 is detected by a spectrometer 5, and then an output of a high-frequency power supply 2 may be subjected to feedback control by a control board 6 based on the signal.

In this manner, the target processing organism 101 floating in the air can be inactivated. Note that, although the floating target sometimes passes through the processing box being incompletely inactivated during one passing through the processing box, by installing the processing box to an air outlet or the like of an air conditioner in the BCR, the air in the room are sure to pass through the inside of the processing box 103 and so every target processing organisms 101 is inactivated after repeating passing through the processing box 103.

Further, it is more preferable that light emission information of the target processing organism 101 floating in the BCR is output from the spectrometer 5 and the amount of air flow of the air convection apparatus in the BCR is controlled. For example, when the target processing organism 101 is increased, increasing the high-frequency power supply 2 or the amount of air flow of the air convection apparatus can kill the target processing organism 101 in BCR at a higher speed.

Note that, upon generation of plasma, the above-described plasma sterilization apparatuses in FIG. 1 and FIGS. 3 to 8 are considered to generate a minute amount of substances harmful to human body such as ozone, nitride oxides, hydrocarbons etc. in addition to oxygen radicals. Thus, load on human body of the plasma flow after irradiation on the target processing organism can be reduced after flowing the plasma flow through harm-eliminating means such as a filter for the deleterious substances mentioned above and then discharging it to the atmosphere.

As described in the foregoing, according to the present invention, it is possible to irradiate plasma only on necessary portions by detecting presence of bacteria in a sterilization processing using plasma. In addition, it is possible to decide irradiation time by determining inactivation and to sterilize the entire BCR room highly efficiently. In this manner, it is only necessary to perform the sterilization work targeting on bacteria in an active state in the BCR and thus it is not necessary to completely stop operation of the BCR for the sterilization processing. In addition, the output power of the power supply can be increased for generating plasma at portions where bacteria in an active state are present and thus sterilization inside the BCR can be performed at low power.

Further, the sterilization technology of surface-attached bacteria using plasma suggested by the present invention is targeted on in-room sterilization of mainly BCRs for regenerative medicine; however, it can be diverted to manufacturing facilities of medical supplies and food supplies and hospital facilities which require elimination of microorganisms. Moreover, it can be also diverted to sterilization of floating bacteria as well as surface-attached bacteria and thus it can be applied to sterilization in homes, refrigerators and so forth for domestic home appliances etc.

### EXPLANATION OF SYMBOLS

1 . . . Insulator
2 . . . High-frequency power supply
3 . . . High-frequency electrode
3′ . . . Ground electrode
4 . . . Plasma
5 . . . Spectrometer
6 . . . Control board
7 . . . Ground electrode for target processing surface
8 . . . Obstacle
9 . . . Display means
100 . . . Target processing surface
101 . . . Target processing organism
102 . . . Autonomous walking plasma sterilization apparatus
103 . . . Processing box
104 . . . Easily contaminated portion
105 . . . Air convection apparatus
106 . . . Intensity of air convection

1. A plasma sterilizer comprising:
a power supply outputting alternating-current voltage;
a plasma source driven by the power supply;
a light emission intensity detector unit detecting light emission intensity of hydrogen or hydroxyl group from a region in which gas that is radicalized by the plasma source is present; and
a controller unit controlling an output of the power supply based on the light emission intensity.

2. The plasma sterilizer according to claim 1,
wherein the controller unit performs control of reducing the output of the power supply more when the light emission intensity is lower than a certain value than when the light emission intensity is higher than the certain value.

3. The plasma sterilizer according to claim 1,
wherein the plasma source further includes a high-frequency electrode and a ground electrode to which the alternate-current voltage is applied for generating plasma.

4. The plasma sterilizer according to claim 3,
wherein the plasma source further includes an insulating film formed to at least one of the high-frequency electrode and the ground electrode.

5. The plasma sterilizer according to claim 1,
wherein the power supply changes an output of a potential or frequency in accordance with an input of a signal from the controller unit.

6. The plasma sterilizer according to claim 1,
wherein the plasma source performs glow discharge.

7. The plasma sterilizer according to claim 1,
wherein the plasma source discharges in the atmosphere to generate oxygen radicals.

8. The plasma sterilizer according to claim 7,
wherein the plasma source further includes an air convection unit for feeding oxygen into the plasma source.

9. The plasma sterilizer according to claim 1,
wherein the light emission intensity detector unit further includes a spectrophotometer capable of detecting a spectrum in the visible region.

10. The plasma sterilizer according to claim 1,
wherein the light emission intensity detector unit detects light emission intensity of phosphorus.

11. The plasma sterilizer according to claim 1,
wherein the plasma source further includes a suction unit for suctioning bacteria and dust.

12. A plasma sterilizer system comprising:
a power supply outputting alternating-current voltage;
a plasma source driven by the power supply;
a light emission intensity detector unit detecting light emission intensity of hydrogen or hydroxyl group from a region in which gas that is radicalized by the plasma source is present;
a clock defining a detection time of the light emission intensity; and
a controller unit controlling an output of the power supply based on the light emission intensity within a certain period measured by the clock.

13. The plasma sterilizer system according to claim 12, further comprising:
an air convection apparatus convecting the air in a bioclean room; and
a controller unit performing control of reducing an amount of air flow of the air convection apparatus more when the light emission intensity is lower than a certain value during a certain period measured by the clock than when the light emission intensity is higher than the certain value.

14. A method of plasma sterilization using: a power supply outputting alternating-current voltage; a plasma source driven by the power supply; a light emission intensity detector unit detecting light emission intensity; and a controller unit performing control of changing an output of the power supply,
the method comprising:
a first step of applying the output of the power supply to the plasma source;
a second step of generating gas that is radicalized by the plasma source;
a third step of detecting light emission intensity of hydrogen or hydroxyl group emitted from a region in which the gas is present; and
a fourth step of controlling the output of the power supply based on the light emission intensity.

15. The method of sterilization according to claim 14, further comprising
a fifth step of detecting light emission intensity of phosphorus emitted from the region in which the gas is present.
